(19) United States
(12) Patent Application Publication
(10) Pub. No.: US 2008/0200750 A1
(43) Pub. Date: Aug. 21, 2008

(54) Polymer encapsulation for medical device

(76) Inventor: Natalie JAMES, Mosman (AU)

Correspondence Address: DANIEL B. SCHEIN, PH.D., ESQ., INC., P. O. BOX 68128, Virginia Beach, VA, 23471, US

(21) Appl. No.: 11/981,818

(22) Filed: Oct. 31, 2007

(30) Foreign Application Priority Data
Nov. 17, 2006 (AU) 2006906443

(51) Int. Cl. A61M 1/12; A61K 9/22

(52) U.S. Cl. 600/16; 604/890.1

(57) ABSTRACT

A rotary blood pump comprising an impeller suspended hydrodynamically within pump housing by thrust forces generated by said impeller during movement in use of said impeller as it rotates about an impeller axis, and the driving torque of said impeller is derived from the magnetic interaction between permanent magnets within the blades of said impeller and windings within said housing, and wherein said windings are encapsulated by a first fluid resistant polymer material, and said housing is at least partially made of a second polymer material that encapsulates said first polymer material.

### TECHNICAL FIELD

The present invention relates to an improved polymer encapsulation for medical device.

### BACKGROUND

Many implantable medical devices have been previously constructed from polymeric materials. Also many of these implantable medical devices include components that are potentially toxic, if these components are allowed to corrode, degrade or oxidize, which commonly occurs to iron, copper based alloys and magnet materials used in implanted electrical and mechanical components.

There has been a long felt need for an implantable medical device primarily constructed of polymeric components that also includes corrodible components, such as iron or copper, configured in a manner to prevent corrosion.

One of the most commonly used polymers in the construction of implantable medical devices is polyether ether ketone (‘PEEK’) because of its biocompatibility, excellent chemical resistance and mechanical strength. However PEEK has a low level of fluid permeability which may induce corrosion within encapsulated metal components of permanently implanted devices.

In U.S. Pat. No. 6,623,475—Siess et al, an active implantable medical device is described in the form of a centrifugal rotary blood pump. The described blood pump includes an impeller that is preferably constructed of plastic or polymeric material within permanent rare-earth magnets. The materials of the pump housing are not discussed in detail in the '475 disclosure, however it is usual for most blood pumps to be constructed from biocompatible metals such as titanium alloys. According to this disclosed configuration of '475, non-biocompatible or toxic components such as the permanent magnets within the impeller may potentially leak toxic compounds into the patient's circulatory system because the corrodible components are not safely encapsulated.

The driving means for rotating the impeller of the disclosed device in '475 relies on a mechanically rotating disc magnetically coupled to the impeller to impart torque force. A greatly improved system is described in U.S. Pat. No. 6,227,797—Watterson et al wherein a rotary blood pump is driven by electromagnet stator coils. However, in the '797 the housing and the impeller are constructed from machined titanium alloy. U.S. Pat. No. 5,536,583—Roberts et al describes a method for coating a metal substrate with a fluoropolymer to act as a barrier to resist corrosion. However the described invention does not describe a second coating layer of a second biocompatible polymer nor does it teach that the method is suitable for use with medical devices (implantable or otherwise).

U.S. Pat. No. 4,897,439—Rau et al describes a coating suitable for coating metals that includes fluoropolymer resin mixed with a polyether resin and an additive, wherein the additive is a material to alter the melting point of the coating. This patent does not teach that the coating may be used with medical devices. Also mixing fluoropolymers with poly ether polymers may not generate the most desired result as the resultant coating may lose some of the properties of both polymers.

U.S. Pat. No. 5,725,519—Penner et al describes a medical device for loading a stent on a balloon catheter. The tube forming part of the medical device is coated with a Teflon™ (a type of fluorocarbon) coating.

U.S. Pat. No. 6,773,815—Amouroux describes a coated metal substrate wherein the metal substrate is coated with a primer, a binder and a fluoropolymer to protect the metal substrate from exposure to high corrosion environments. '815 patent draws particular attention to the use of the coating in applications to offshore hot oil wells but makes no reference to use in medical applications or in-situ environments. This process or application may not be suitable for implantable medical devices or applications.

The present invention aims to address or at least ameliorate one or more of the disadvantages associated with the above mentioned prior art.

### SUMMARY OF THE INVENTION

According to a first aspect the present invention consists in an implantable medical device, wherein said medical device includes non-biocompatible or toxic components encapsulated with a first polymer and wherein said first polymer is injection moulded within a second polymer.

Preferably, said first polymer is relatively fluid resistant to prevent fluid ingress.

Preferably, said non-biocompatible or toxic components include metals that are prone to corrosion or toxic leakage when implanted within a patient.

Preferably, said implanted medical device requires a power source to function.

Preferably, said first polymer is a fluoropolymer; parylene; or paralene. Preferably, said second polymer is poly ether or PTFE.

Preferably, said first polymer has a higher melting point than said second polymer.

According to a second aspect the present invention consists of a rotary blood pump wherein said pump comprises a housing with a cavity, wherein said cavity includes a rotor with an impeller and wherein impeller includes several permanent magnets, when in use, said impeller is magnetically urged to rotate by use of a single set of electromagnetic stator coils mounted below the impeller within the housing.

Preferably, the set of electromagnetic stator coils are encapsulated with a fluid resistant polymer to prevent component leakage.

Preferably, said a yoke is encapsulated within the polymeric housing and mounted above the impeller.

According to a third aspect the present invention consists of a rotary blood pump comprising an impeller suspended hydrodynamically within a pump housing by thrust forces generated by said impeller during movement in use of said impeller as it rotates about an impeller axis, and the driving torque of said impeller is derived from the magnetic interaction between permanent magnets within the blades of said impeller and windings within said housing, and wherein said windings are encapsulated by a first fluid resistant polymer material, and said housing is at least partially made of a second polymer material that encapsulates said first polymer material.

Preferably, said windings comprise a single set of coils disposed within the base of said housing.

Preferably, said impeller is at least partially made from a polymer material.

Preferably, said impeller is made from said second polymer material. Preferably in one embodiment said housing includes a yoke.

### BRIEF DESCRIPTION OF THE DRAWINGS

Embodiments of the present invention will now be described with reference to the accompanying drawings wherein:

FIG. 1 depicts an exploded cross-sectional side view of a first preferred embodiment of the present invention;

FIG. 2 depicts an exploded cross-sectional side view of a second preferred embodiment of the present invention;

FIG. 3 depicts a top view of an enlarged portion of the second preferred embodiment; and

FIG. 4 depicts an exploded cross-sectional side view of a third preferred embodiment of the present invention.

### BRIEF DESCRIPTION OF THE PREFERRED EMBODIMENTS

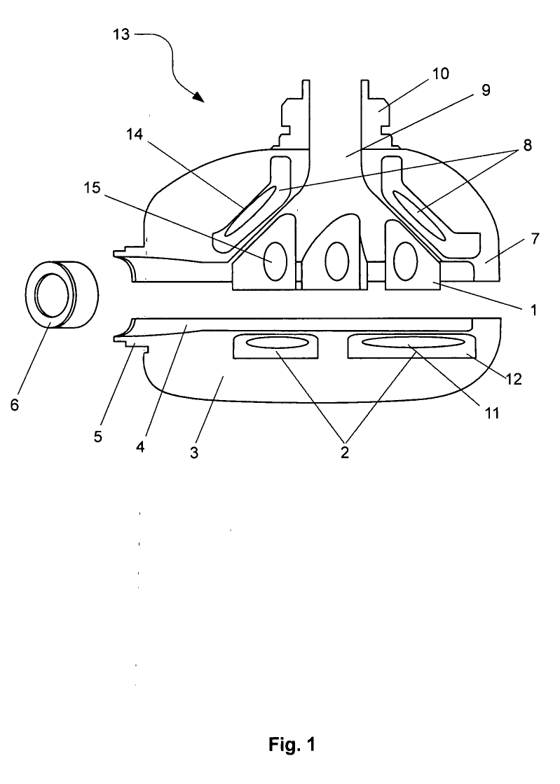

The first preferred embodiment of the present invention depicted in FIG. 1 shows a rotary and centrifugal implantable blood pump 13 suitable for long term implantation within the body of a patient. The preferred blood pump 13 is similar to the left ventricle assist device described within U.S. Pat. No. 6,227,797—Watterson et al.

The blood pump 13 of the first preferred embodiment is of a centrifugal design including an impeller 1 adapted to rotate, when in use, within a cavity 4 formed within a housing and preferably pumps blood from the inlet 9 to the outlet 5. In this embodiment, the impeller 1 is hydrodynamically borne or suspended, when rotated, by thrust forces generated by the use of wedge shaped restrictions formed between the outer edges of the blades of the impeller 1 interacting with the internal walls of the cavity 4. The housing, in this preferred embodiment, comprises of upper 7 and lower 3 housing portions, which are both preferably constructed of a polymeric material.

Preferably, the housing portions 3 and 7 are constructed of injection moulded PEEK or injection moulded PTFE. Additives may be mixed at low concentration with the PEEK or PTFE resin prior to moulding to facilitate hermetic bonding processes at a later stage of manufacture. A range of polymer additives has been developed which create near infrared absorbent pigments for transmission laser welding. These include products produced by BASF Group (Lumogen™), Gentex Corp (Clearweld™) and Merck (Iriodin™).

In this embodiment, the upper housing 7 includes an upper stator assembly 8 and the lower housing 3 includes a lower stator assembly 2. The stator assemblies 8 and 2, in this embodiment, are mounted on opposite sides of the impeller 1 within their respective housing portion 3 and 7. The inclined angle of the upper stator assembly 8 allows for torque force to be applied to the impeller 7 in both the axial and radial direction in respect of the impeller's axis of rotation. The lower stator assembly 2, in this embodiment, is only acting in the axial direction relative to the axis of rotation of the impeller 7.

Each of the stator assemblies 2 and 8 comprise three electromagnetic coils (or windings) 11, which are constructed of an electrically conductive material such as copper, iron or Litz wire. The electrically conductive materials, used in the coils, are generally materials that may be potentially toxic to patients, if they leach into the patient body's and there may also be deleterious effects to the functionality of the blood pump, if they corrode or oxidize. For electrical insulation and protection, the copper coil wires are coated with polyimide, however this coating is not a moisture protective coating.

Preferably, the coils 11 are encapsulated with a first fluid resistant polymer 12 such as a fluoropolymer. The most preferred fluoropolymers are perfluoroalkoxyethylene (PFA) and polytetrafluoroethylene (‘PTFE’) which is commonly known by the brand name Teflon™, which is a trademark owned by Dupont. Other fluoropolymers may also achieve the same or similar results and these include fluorinated ethylene propylene (‘FEP’). Fluoropolymers are generally easily formable and generally more fluid resistant than other polymers due to hydrophobic properties. Additionally, various chemical additives may be added to the fluoropolymer layer 12 to further increase its impermeability to fluids.

In this first embodiment, the impeller is preferably constructed from a titanium alloy. The advantage with using a titanium alloy is that it has the mechanical stability, biocompatibility and general dimension stability which are critical for use with hydrodynamic bearings on the outer edges of the impeller 1. The impeller 1 preferably includes four blades mounted in a general circular arrangement and each of the blades has a general shark fin shape, as per FIG. 1. Each blade includes a permanent magnet 15 which interacts with the energised stator coils 2 and 8 in the base and upper housing portions 3 and 7. Typically, the permanent magnets 15 are constructed from neodymium-iron-boron magnets or rare earth magnets. In addition, the use of titanium alloy at approx 500 micron thickness allows for the blade enclosures to maximise the volume of magnet material enclosed, which contributes to motor efficiency.

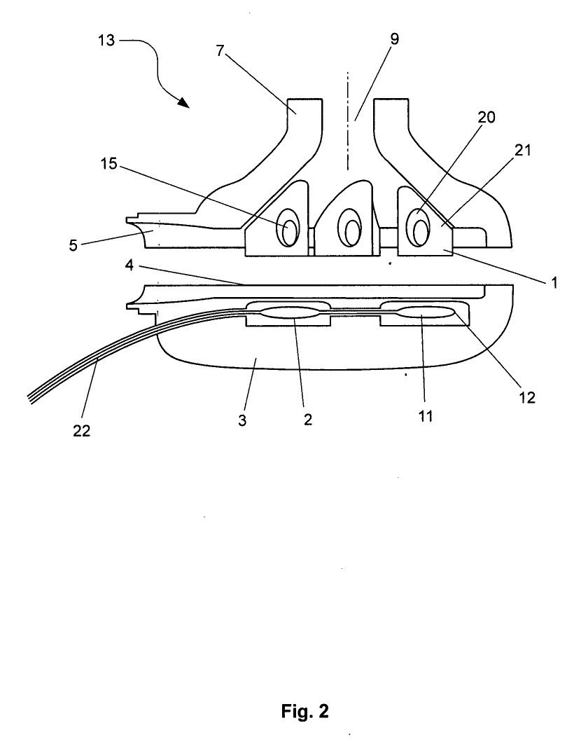

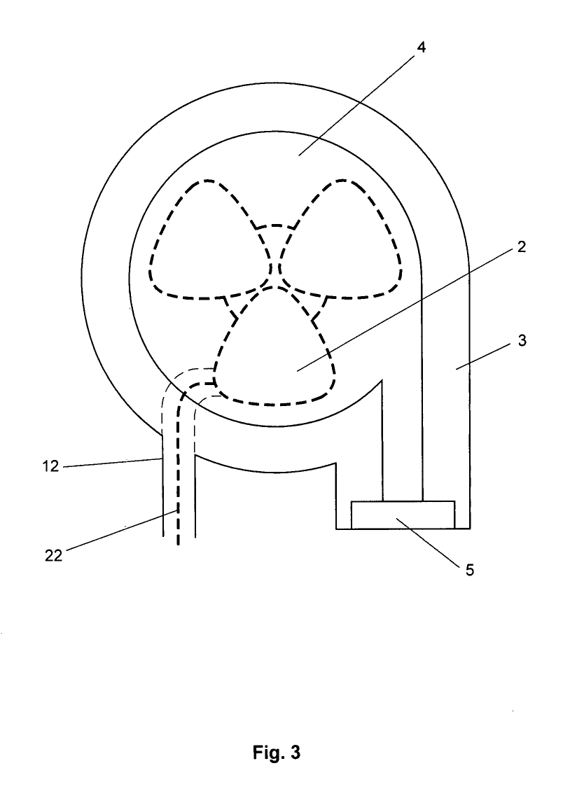

A second preferred embodiment of the present invention is depicted in FIGS. 2 and 3. FIG. 2 depicts an injection moulded blood pump 13 constructed from mainly polymeric materials wherein the blood pump 13 is similar to the first preferred embodiment depicted in FIG. 1. The blood pump 13 depicted in the second preferred embodiment does not include the upper stator assembly 8. The removal of the upper stator assembly greatly simplifies the overall construction and manufacture of the blood pump 13 and may significantly reduce the implanted volume of the pump and the associated manufacturing costs for such a pump 13.

In FIG. 2, all of the electronics have been limited to the lower base housing portion 3. This feature allows all of the wiring within the blood pump 13 to be injection moulded in one step process. Preferably, all of the wires and lower stator assembly 2 are encapsulated within a first polymer 12 that is relatively fluid resistant. The housing portions 3 and 7 are injection moulded from a second polymer such as PEEK or PTFE and the second polymer encapsulates the first polymer.

The fluid resistant nature of the first polymer 12 may generally prevent corrosion or degradation of the wires and the lower stator assembly 2.

Further, in the second preferred embodiment it may no longer be required to use feed-through technology to connect the internal encapsulated wires to the outside of the pump 13. The encapsulated wires may be allowed to simply extend through the wall of the lower housing portion 3, as depicted in FIG. 3. Specifically, FIG. 3 depicts a top view of a portion of the second preferred embodiment. Said portion is the lower housing 3 and in FIG. 3, the lower housing 3 is represented as being relatively transparent to depict the direct connection between the percutaneous lead and the coils of the motor stator. In FIG. 3 the setup of the wiring is shown wherein three stator coils which form the lower stator assembly 2 are joined by wiring. The wiring and the stator coils are all encapsulated with a first polymer which is preferably a fluoropolymer including but not limited to erfluoroalkoxyethylene or polytetrafluoroethylene. Alternatively, feedthrough technology may be used, where there are manufacturing or other advantages.

In this second preferred embodiment, the impeller 1 may be constructed from titanium alloy, as described for the first preferred embodiment.

A third embodiment would be to construct the rotor from polymer in a similar manner to the housing. The permanent magnets 15 are encapsulated within each blade of the impeller 1. Since the magnet material is prone to corrosion, an option would be to coat the permanent magnets 15 with parylene prior to encapsulation to provide a thin, highly conformal, pin-hole free moisture-resistant barrier. Forms of parylene are stable at relatively high temperatures (melting point 400° C. and above) and biocompatible. They would thus be stable when exposed to further injection moulding steps. For further moisture protection, the magnet, in the parylene-coated or the non-coated form, is then encapsulated within a layer of the first polymer 20 which is relatively fluid resistant and the first polymer is injection moulded within a second polymer, which may be a poly-ether 21 or PTFE. Additives may be mixed with the second polymer to increase its dimensional stability and to prevent warping or dimensional distortion. A further option would be to parylene coat the magnet material in sufficient thickness of parylene for adequate corrosion resistance and then injection mould this coated magnet within the second polymer, which may be a poly-ether 21 or PTFE. This polyether or PTFE would provide the required materials properties, including low friction coefficient, biocompatibility, dimensional stability, mechanical strength and corrosion resistance, as required for the interface with the blood, and with the housing 13 of the pump.

An alternative approach for production of the magnet materials for the third embodiment is to “compression mould” the magnet material using an epoxy with high temperature tolerance as the binder, or alternatively to injection mould the magnet material. Moulded magnets may be parylene-coated for additional corrosion resistance, if required, prior to injection moulding within a second polymer, which may be a polyether 21 or PTFE.

Preferably, the first polymer barrier to moisture penetration should include a melting point characteristic which is relatively higher than the second polymer. This may allow the second polymer to be injection moulded around the first polymer.

Preferred first fluoropolymers may include PFA, or fluorinated ethylene propylene (‘PEP’), which generally have a melting point (‘MP’) around 310° C. to 260° C., respectively. A second preferred polymer, PEEK, generally has a MP of 334° C. This means that the second polymer has a greater MP than the first polymer and if the second polymer was injection moulded around the first polymer, the first polymer may experience melting. There are several methods that may be used to prevent or limit the melting. The first method is to make the encapsulation of the first polymer relatively thick and increase the cooling time of the second polymer, this will reduce the effective damage done to the first polymer. A second method may be to increase the MP of the first polymer and/or reduce the MP of the second polymer, or to include polymer additives to adjust the MP accordingly.

An additional alternative for polymeric coating for moisture resistance is the use of a high temperature stable paralene coating as the primary barrier for moisture penetration. Since parylene has a melting point at 400° C. and above it would readily tolerate enclosure by injection moulding of the fITst fluoropolymer, (PFA, PTFE or PEP) and then the second polymer, polyether. Moisture resistance and other protection characteristics provided by the paralene barrier may be able to be sufficient to paralene-coat the magnet material with a coating of sufficient thickness for corrosion protection without the fluoropolymer coating, and then injection mould directly into the second polymer, the polyether or PTFE.

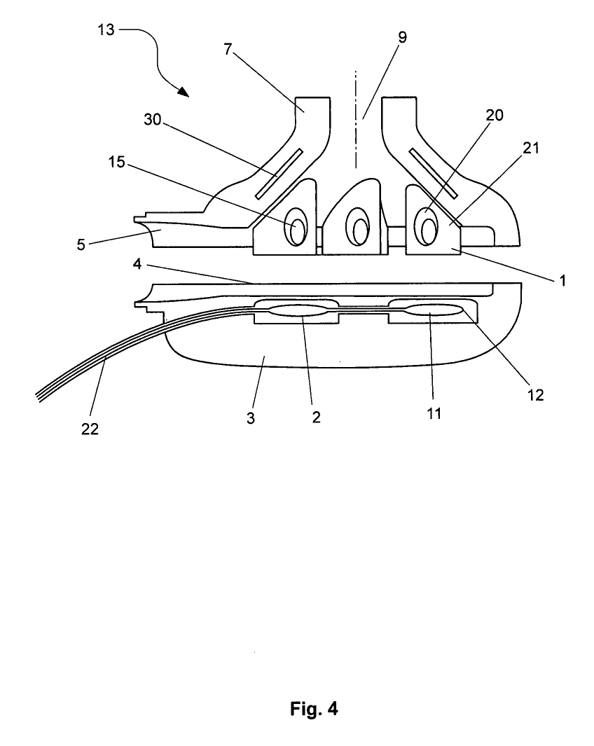

Preferably, a further fourth preferred embodiment of the present invention (as shown in FIG. 4) may include a ferromagnetic yoke 30 to replace the upper stator assembly of the first preferred embodiment depicted in FIG. 1. The removal of the upper stator assembly, as per FIG. 2, may reduce the manufacturing complexity and cost.

However, the removal of the upper stator assembly may also reduce the efficiency of the DC brushless motor formed within the blood pump 13. To counteract this effect, the inclusion of a yoke 30 encapsulated within the upper housing 7 may increase the motor efficiency. Preferably, the yoke 30 may also be encapsulated within a first polymer, including paralene or a fluoropolymer (not shown) which is in turn injection moulded within a second polymer that forms the upper housing 7. The yoke 30 may be configured in a general ring shape or an annulus mounted above the impeller 1 within or on the housing and may be mounted on an opposed side of the impeller 1 as compared with the lower stator assembly 2. The yoke 30 may be constructed of: permalloy; ferrite, an iron alloy, or a powdered metal alloy with suitable magnetic properties.

The above descriptions detail only some of the embodiments of the present invention. Modifications may be obvious to those skilled in the art and may be made without departing from the scope and spirit of the present invention.

1. An implantable medical device, wherein said medical device includes non-biocompatible or toxic components encapsulated with a first polymer and wherein said first polymer is injection moulded within a second polymer.

2. The implantable medical device as claimed in claim 1, wherein said first polymer is relatively fluid resistant to prevent fluid ingress.

3. The implantable medical device as claimed in claim 2, wherein said non-biocompatible or toxic components include metals that are prone to corrosion or toxic leakage when implanted within a patient.

4. The implantable medical device as claim in claim 3, wherein said implanted medical device requires a power source to function.

5. The implantable medical device as claimed in claim 4, wherein said first polymer is selected from the group consisting of a fluoropolymer, parylene, and paralene.

6. The implantable medical device as claimed in claim 5, wherein said second polymer is poly ether or PTFE.

7. The implantable medical device as claimed in claim 1, wherein said first polymer has a higher melting point than said second polymer.

8. A rotary blood pump wherein said pump comprises a housing with a cavity, wherein said cavity includes a rotor with an impeller and wherein said impeller includes several permanent magnets, wherein when in use said impeller is magnetically urged to rotate by use of a single set of electromagnetic stator coils mounted below the impeller within the housing.

9. The rotary blood pump as claimed in 8, wherein said set of electromagnetic stator coils are encapsulated with a fluid resistant polymer to prevent component leakage.

10. The rotary blood pump as claimed in claim 8, wherein a yoke is encapsulated within the housing and mounted above the impeller.

11. A rotary blood pump comprising an impeller suspended hydrodynamically within a pump housing by thrust forces generated by said impeller during movement in use of said impeller as it rotates about an impeller axis, and the driving torque of said impeller is derived from the magnetic interaction between permanent magnets within the blades of said impeller and windings within said housing, and wherein said windings are encapsulated by a first fluid resistant polymer material, and said housing is at least partially made of a second polymer material that encapsulates said first polymer material.

12. A rotary blood pump as claimed in claim 11, wherein said windings comprise a single set of coils disposed within the base of said housing.

13. A rotary blood pump as claimed in claim 11, wherein said impeller is at least partially made from a polymer material.

14. A rotary blood pump as claimed in claim 11, wherein said housing includes a yoke.

15. A rotary blood pump as claimed in claim 14, wherein said impeller is made from said second polymer material.

16. A rotary blood pump as claimed in claim 12, wherein said impeller is at least partially made from a polymer material.

17. A rotary blood pump as claimed in claim 12, wherein said housing includes a yoke.

18. A rotary blood pump as claimed in claim 13, wherein said housing includes a yoke.

19. A rotary blood pump as claimed in claim 17, wherein said impeller is made from said second polymer material.

20. A rotary blood pump as claimed in claim 18, wherein said impeller is made from said second polymer material.
